SEQUENCE LISTING (6 sequences)

SEQ ID NO: 1 - DNA, 3935 bp, Corynebacterium glutamicum
  CDS (2022)..(3851)
ctggcagttctcctaattgatcgcgggaattatcagaaatagacattatttgttatttttcctgttcaactttaaaacttcaatattcgtgagtttggatgaatccctagagcactaccttttagacctctcgctgcaatttaggccagttgagatttaagctttccgacgattcttctcattactgcaatcgtaccggcgatggtggacacgatgacatgaaagagcattaaagcaatcaagtacaggctgaagtagttaaaccactccactccggtgctctgtgataaaaaatgcgcacccaaactcaaagtgccaactgggaaggtactggcccaccatgtggggctgtatgtcgcccctttgaaaacagctctgtagaacacaaagtgagcgatggctcccagaggaatcgtaaaaattcccatgatgatgccgtaaataatgcccattgtgattgctgtcttggatccaaaggacgcaccgatgagctgagctgctgcagtggattggcccaccatacccaaaggaatccatgatgttggtgttgccatcagtgggatgccctgcgccttggggccgaaatagtagaaatacactcgggtaaaaactgctggtgcagacgccaaagttaaaaggaagagcccgaaagaaacccacagcatcgccggaagttcaaagtgctcatggagttgtgctgccgaggtggaagcaaccatcggcgtgacaagaggaagaccccacgcaaaagttggtgtgcccgccttagatcgcaaaatggccgttatatataaggaataggcaacaagtcccacggctgtgccaatagaccagcacacaaacataaatccccacagatcatcacccaaaactacggggcttgcagttcccaatgcgatcaaacccatggacagcattgcccatgccggcatgacttcagttttgaatgaaggagagcggtagattagccaaccgccaataatgacaattgccaccacaacagctaacgcgaagaagaaatctgcgacgactggaaaaccatggattttcaacagtgatgacaacaatgagatgcccatgagggaaccagcccacgaggggccaggtggaggtaagaccgcagcgtagcttttggtcgaagaaggagtgggcatgcccattactttaagcctttggggcagtgaaaccgctaaatgggagcgttgtgcgctcgatcactggtctagacctttgggctccaaaagttgcaatttcgcgaatacttcaacacttgtttgcaatgtttgttaataaatgggttcgctagtggattctgtcgttagtactggccgtcgtggtggggtcatgtatttaggtagggcaaagttaagatcagagcactttttgatacgactaactggatataacctttggggtaacgtggggatgtgtgtgagtaattttcaaagtatttaaaagggggatctagggtaaaaatttggcttcaagtacatatctttagttcggtagttgagggcgggtggtgacagtgcgggcatgcatgtgagtgtaaatgttgttttaaaaaggtgtgtactgacagtgggccggtttgtgctggtcggccactagcggagtgcttggattgtgatggcagggtaagggaaagggattaccattaccgctgttcttggcgttttgttgcctattgtccgaatgttaagtgttaatggtgggaaaactgggaaagttgtcccctggaatgtgtgagaattgcccaaatctgaacccaatggccatggacggggaatgaactgtcggagaacggttgaggttaattcttgaaaccacccccaaaataggctatttaaacgggtgctctcatattaaagaaagtgtgtagatgcgtgtgggcagggggtaggtccactggtaatgacaaatgtgtccgttgtctcacctaaagttttaactagttctgtatctgaaagctacgctagggggcgagaactctgtcgaatgacacaaaatctggagaagtaatgactactgctgcaatcaggggccttcagggcgaggcgccgaccaagaataaggaactgctgaactggatcgcagacgccgtcgagctcttccagcctgaggctgttgtgttcgttgatggatcccaggctgagtgggatcgcatggcggaggatcttgttgaagccggtaccctcatcaagctcaacgaggaaaagcgtccgaacagctacctagctcgttccaacccatctgacgttgcgcgcgttgagtcccgcaccttcatctgctccgagaaggaagaagatgctggcccaaccaacaactgggctccaccacaggcaatgaaggacgaaatgtccaagcattacgctggttccatgaaggggcgcaccatgtacgtcgtgcctttctgcatgggtccaatcagcgatccggaccctaagcttggtgtgcagctcactgactccgagtacgttgtcatgtccatgcgcatcatgacccgcatgggtattgaagcgctggacaagatcggcgcgaacggcagcttcgtcaggtgcctccactccgttggtgctcctttggagccaggccaggaagacgttgcatggccttgcaacgacaccaagtacatcacccagttcccagagaccaaggaaatttggtcctacggttccggctacggcggaaacgcaatcctggcaaagaagtgctacgcactgcgtatcgcatctgtcatggctcgcgaagaaggatggatggctgagcacatgctcatcctgaagctgatcaacccagagggcaaggcgtaccacatcgcagcagcattcccatctgcttgtggcaagaccaacctcgccatgatcactccaaccatcccaggctggaccgctcaggttgttggcgacgacatcgcttggctgaagctgcgcgaggacggcctctacgcagttaacccagaaaatggtttcttcggtgttgctccaggcaccaactacgcatccaacccaatcgcgatgaagaccatggaaccaggcaacaccctgttcaccaacgtggcactcaccgacgacggcgacatctggtgggaaggcatggacggcgacgccccagctcacctcattgactggatgggcaacgactggaccccagagtccgacgaaaacgctgctcaccctaactcccgttactgcgtagcaatcgaccagtccccagcagcagcacctgagttcaacgactgggaaggcgtcaagatcgacgcaatcctcttcggtggacgtcgcgcagacaccgtcccactggttacccagacctacgactgggagcacggcaccatggttggtgcactgctcgcatccggtcagaccgcagcttccgcagaagcaaaggtcggcacactccgccacgacccaatggcaatgctcccattcattggctacaacgctggtgaatacctgcagaactggattgacatgggtaacaagggtggcgacaagatgccatccatcttcctggtcaactggttccgccgtggcgaagatggacgcttcctgtggcctggcttcggcgacaactctcgcgttctgaagtgggtcatcgaccgcatcgaaggccacgttggcgcagacgagaccgttgttggacacaccgctaaggccgaagacctcgacctcgacggcctcgacaccccaattgaggatgtcaaggaagcactgaccgctcctgcagagcagtgggcaaacgacgttgaagacaacgccgagtacctcactttcctcggaccacgtgttcctgcagaggttcacagccagttcgatgctctgaaggcccgcatttcagcagctcacgcttaaagttcacgcttaagaactgctaaataacaagaaaggctcccaccgaaagtgggagcctttcttgtcgttaagcgatgaatt

SEQ ID NO: 2 - protein, 610 aa, Corynebacterium glutamicum
MTTAAIRGLQGEAPTKNKELLNWIADAVELFQPEAVVFVDGSQAEWDRMAEDLVEAGTLIKLNEEKRPNSYLARSNPSDVARVESRTFICSEKEEDAGPTNNWAPPQAMKDEMSKHYAGSMKGRTMYVVPFCMGPISDPDPKLGVQLTDSEYVVMSMRIMTRMGIEALDKIGANGSFVRCLHSVGAPLEPGQEDVAWPCNDTKYITQFPETKEIWSYGSGYGGNAILAKKCYALRIASVMAREEGWMAEHMLILKLINPEGKAYHIAAAFPSACGKTNLAMITPTIPGWTAQVVGDDIAWLKLREDGLYAVNPENGFFGVAPGTNYASNPIAMKTMEPGNTLFTNVALTDDGDIWWEGMDGDAPAHLIDWMGNDWTPESDENAAHPNSRYCVAIDQSPAAAPEFNDWEGVKIDAILFGGRRADTVPLVTQTYDWEHGTMVGALLASGQTAASAEAKVGTLRHDPMAMLPFIGYNAGEYLQNWIDMGNKGGDKMPSIFLVNWFRRGEDGRFLWPGFGDNSRVLKWVIDRIEGHVGADETVVGHTAKAEDLDLDGLDTPIEDVKEALTAPAEQWANDVEDNAEYLTFLGPRVPAEVHSQFDALKARISAAHA

SEQ ID NO: 3 - DNA, 20 bp, Artificial Sequence
  Description of Artificial Sequence Primer
ggaactgctgaactggatcg

SEQ ID NO: 4 - DNA, 20 bp, Artificial Sequence
  Description of Artificial Sequence Primer
gaactggctgtgaacctctg

SEQ ID NO: 5 - DNA, 20 bp, Artificial Sequence
  Description of Artificial Sequence Primer
catctcacgctatgaattct

SEQ ID NO: 6 - DNA, 18 bp, Artificial Sequence
  Description of Artificial Sequence Primer
acgaggcctgcagcaata

(19) United States
(12) Patent Application Publication
(10) Pub. No.: US 2002/0042104 A1
(43) Pub. Date: Apr. 11, 2002

(54) Process for the fermentative preparation of D-pantothenic acid using coryneform bacteria

(76) Inventors: Nicole Dusch, Bielefeld (DE); Georg Thierbach, Bielefeld (DE)

Correspondence Address: Pillsbury Winthrop LLP, Intellectual Property Group, East Tower, 1100 New York Avenue, N.W., Ninth Floor, Washington, DC, 20005-3918, US

(21) Appl. No.: 09/852,118

(22) Filed: May 10, 2001

(30) Foreign Application Priority Data
May 30, 2000 (DE) DE 100 26 758.0

(51) Int. Cl. C12P 13/04; C12N 9/12; C12N 1/21

(52) U.S. Cl. 435/106; 435/194; 435/252.3

(57) ABSTRACT

The invention provides a process for preparing D-pantothenic acid by the fermentation of coryneform bacteria in which bacteria are used in which the nucleotide sequence (pck gene) coding for phosphoenolpyruvate carboxykinase (EC 4.1.1.49) is attenuated, wherein the following steps are performed:

a) fermentation of D-pantothenic acid producing bacteria in which at least the gene coding for phosphoenolpyruvate carboxykinase is attenuated,

b) enrichment of D-pantothenic acid in the medium or in the cells of the bacteria, and

c) isolation of the D-pantothenic acid produced.

The invention provides a process for the fermentative preparation of D-pantothenic acid using coryneform bacteria in which the pck gene is attenuated.

### PRIOR ART

Pantothenic acid is a commercially important vitamin which is used in cosmetics, medicine, human nutrition and animal nutrition.

Pantothenic acid can be prepared by chemical synthesis or biotechnically by the fermentation of suitable microorganisms in appropriate liquid nutrient media. DL-pantolactone is an important intermediate in the case of chemical synthesis. It is prepared in a multistage process from formaldehyde, isobutylaldehyde and cyanide. In further process steps, the racemic mixture is resolved, D-pantolactone is condensed with β-alanine and the desired D-pantothenic acid is obtained in that way.

The advantage of fermentative preparation by microorganisms is direct formation of the desired stereoisomeric D-form which does not contain any L-pantothenic acid.

Various species of bacteria such as e.g. Escherichia coli, Arthrobacter ureafaciens, Corynebacterium erythrogenes, Brevibacterium ammoniagenes and also yeasts such as e.g. Debaromyces castellii, as shown in EP-A 0 493 060, can produce D-pantothenic acid in a liquid nutrient medium which contains glucose, DL-pantoic acid and β-alanine. EP-A 0 493 060 also demonstrates that the formation of D-pantothenic acid is improved in the case of Escherichia coli by the amplification of pantothenic acid biosynthesis genes from E. coli, which are contained on the plasmids pFV3 and pFV5, in a liquid nutrient medium which contains glucose, DL-pantoic acid and β-alanine.

EP-A 0 590 857 and U.S. Pat. No. 5,518,906 describe mutants derived from Escherichia coli strain IFO3547, such as FV5714, FV525, FV814, FV521, FV221, FV6051 and FV5069, which carry resistance to various antimetabolites such as salicylic acid, α-ketobutyric acid, β-hydroxyaspartic acid, O-methylthreonine and α-ketoisovaleric acid. They produce pantoic acid in a liquid nutrient medium which contains glucose and they produce D-pantothenic acid in a liquid nutrient medium which contains glucose and β-alanine. Furthermore it is shown in EP-A 0 590 857 and U.S. Pat. No. 5,518,906 that after enhancement of the pantothenic acid biosynthesis genes, which are contained on plasmid pFV31, in the strains mentioned above, the production of D-pantoic acid is improved in glucose-containing liquid nutrient media and the production of D-pantothenic acid is improved in a liquid nutrient medium which contains glucose and β-alanine.

Processes for preparing D-pantothenic acid with the aid of Corynebacterium glutamicum are only partly disclosed in the literature. Thus, Sahm and Eggeling (Applied and Environmental Microbiology 65(5), 1973-1979 (1999) report on the effect of overexpression of the genes panB and panC and Dusch et al. (Applied and Environmental Microbiology 65(4), 1530-1539 (1999)) report on the effect of the gene panD on the formation of D-pantothenic acid.

### OBJECT OF THE INVENTION

The inventors have stated that the object is the provision of new principles for improved processes for the fermentative preparation of pantothenic acid using coryneform bacteria.

### DESCRIPTION OF THE INVENTION

The vitamin pantothenic acid is a commercially important product which is used in cosmetics, medicine, human nutrition and animal nutrition. There is a general interest in the provision of improved processes for preparing pantothenic acid.

Whenever D-pantothenic acid or pantothenic acid or pantothenate are mentioned in the following, not only the free acid but also the salts of D-pantothenic acid such as e.g. the calcium, sodium, ammonium or potassium salt are also meant to be included.

The invention provides a process for the fermentative preparation of D-pantothenic acid using coryneform bacteria, in which the nucleotide sequence (pck gene) coding for the enzyme phosphoenolpyruvate carboxykinase (PEP carboxykinase) (EC 4.1.1.49) is attenuated.

Optionally, the strains used already produce D-pantothenic acid before attenuation of the pck gene.

Preferred embodiments may be found in the Claims.

The term “attenuation” in this connection describes the reduction or switching off of the intracellular activity of one or more enzymes (proteins) in a microorganism, which are coded by the corresponding DNA, by using, for example, a weak promoter or a gene or allele which codes for a corresponding enzyme with a lower activity or inactivates the corresponding enzyme (protein) and optionally combines these actions.

The microorganisms which are the object of the present invention can produce D-pantothenic acid from glucose, saccharose, lactose, fructose, maltose, molasses, starch, cellulose or from glycerine and ethanol. They are representatives of coryneform bacteria, in particular the genus Corynebacterium. From the genus Corynebacterium the species Corynebacterium glutamicum is mentioned in articular, this being recognised by specialists for its ability to produce L-amino acids.

Suitable strains of the genus Corynebacterium, in particular of the species Corynebacterium glutamicum, are, for example, the known wild type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Brevibacterium flavum ATCC14067

Brevibacterium lactofermentum ATCC13869 and

Brevibacterium divaricatum ATCC14020

and D-pantothenic acid-producing mutants prepared therefrom such as, for example,

Corynebacterium glutamicum ATCC13032ΔilvA/pEC7panBC

Corynebacterium glutamicum ATCC13032/pND-D2

It was found that coryneform bacteria produce pantothenic acid in an improved way after attenuation of the pck gene coding for phosphoenolpyruvat carboxykinase (PEP carboxykinase) (EC 4.1.1.49).

The nucleotide sequence for the pck gene is given in SEQ ID No 1 and the amino acid sequence of the enzyme protein produced therefrom is given in SEQ ID No 2.

The pck gene described in SEQ ID No 1 is used in accordance with the invention. Furthermore, alleles of the pck gene are used which are produced from the degeneracy of the genetic code or by functionally neutral sense mutations.

To produce an attenuation, either expression of the pck gene or the catalytic properties of the enzyme protein may be reduced or switched off. Optionally, both actions may be combined.

Reduction of gene expression may be performed by suitable culture management or by genetic modification (mutation) of the signal structures for gene expression. Signal structures for gene expression are, for example, repressor genes, activator genes, operators, promoters, attenuators, ribosome bonding sites, the start codon and terminators. A person skilled in the art can find information about these in e.g. patent application Ser. No. WO 96/15246, in Boyd and Murphy (Journal of Bacteriology 170:5949 (1988)), in Voskuil and Chambliss (Nucleic Acids Research 26:3548 (1998), in Jensen and Hammer (Biotechnology and Bioengineering 58:191 (1998)), in Patek et al. (Microbiology 142:1297 (1996) and in well-known textbooks on genetics and molecular biology such as e.g. the textbooks by Knippers (“Molekulare Genetik”, 6th edition, Georg Thieme Verlag, Stuttgart, Germany, 1995) or by Winnacker (“Gene und Klone”, VCH Verlagsgesellschaft, Weinheim, Germany, 1990).

Mutations which lead to modification of or a reduction in the catalytic properties of enzyme proteins are disclosed in the prior art; the articles by Qiu and Goodman (Journal of Biological Chemistry 272:8611-8617 (1997)), Sugimoto et al. (Bioscience Biotechnology and Biochemistry 61:1760-1762 (1997)) and Möckel (“Die Threonindehydratase aus Corynebacterium glutamicum: Aufhebung der allosterischen Regulation und Struktur des Enzyms”, Berichte des Forschungszentrums Jülichs, Jül-2906, ISSN09442952, Jülich, Germany, 1994) may be mentioned as examples. Summarising reviews may be found in known textbooks on genetics and molecular engineering such as e.g. the book by Hagemann (“Allgemeine Genetik”, Gustav Fischer Verlag, Stuttgart, 1986).

Suitable mutations are transitions, transversions, insertions and deletions. Missense mutations or nonsense mutations are referred to, depending on the effect of amino acid exchange on the enzyme activity. Insertions or deletions of at least one base pair in a gene lead to frame shift mutations which then mean that the wrong amino acids are incorporated or the translation is terminated prematurely. Deletions of several codons lead typically to a complete failure in enzyme activity. Instructions for these types of mutations are part of the prior art and may be obtained from known textbooks on genetic and molecular biology such as e.g. the textbook by Knippers (“Molekulare Genetik”, 6th edition, Georg Thieme Verlag, Stuttgart, Germany, 1995), the book by Winnacker (“Gene und Klone”, VCH Verlagsgesellschaft, Weinheim, Germany, 1990) or the book by Hagemann (“Allgemeine Genetik”, Gustav Fischer Verlag, Stuttgart, 1986).

An example of a mutated pck gene is the Δpck allele contained in the plasmid pK19mobsacBΔpck (FIG. 3). The Δpck allele contains only the 5′ and 3′ flanks of the pck gene; a 1071 bp long section of the coding region is missing (deletion). This Δpck allele can be incorporated into coryneform bacteria by integration mutagenesis. The plasmid pK19mobsacBΔpck mentioned above, which is not replicable in C. glutamicum, is used for this purpose. After transfer by conjugation or transformation and homologous recombination by means of a first, integration causing, “cross over” event and a second, excision causing, “cross over” event in the pck gene, the Δpck-allele is incorporated and a total loss of enzyme function is produced in the particular strain involved.

Instructions and explanations relating to integration mutagenesis can be found, for example, in Schwarzer and Pühler (Bio/Technology 9, 84-87 (1991)) or Peters-Wendisch et al. (Microbiology 144, 915-927 (1998)).

An example of a pantothenic acid-producing coryneform bacterial strain with an attenuated pck gene is the strain ATCC13032ΔilvAΔpck/pXT-panD.

In addition, it may be advantageous for the production of pantothenic acid, in addition to attenuating the gene coding for phosphoenolpyruvate carboxykinase, to enhance in particular to overexpress one or more further genes coding for enzymes in the pantothenic acid biosynthetic pathway or the ketoisovaleric acid biosynthetic pathway, such as e.g.

the panB gene coding for ketopantoate hydroxymethyl-transferase (Sahm et al., Applied and Environmental Microbiology, 65, 1973-1979 (1999)) or

the panC gene coding for pantothenate synthetase (Sahm et al., Applied and Environmental Microbiology, 65, 1973-1979 (1999)) or

the ilvD gene coding for dihydroxyacid dehydratase.

Furthermore, it may be advantageous for the production of pantothenic acid, in addition to attenuating phosphoenolpyruvate carboxykinase, to switch off undesired side reactions (Nakayama: “Breeding of Amino Acid Producing Micro-organisms”, in: Overproduction of Microbial Products, Krumphanzl, Sikyta, Vanek (eds.), Academic Press, London, UK, 1982).

The microorganisms prepared according to the invention may be cultivated continuously or in a batch process or in a fed batch or repeated fed batch process for the purposes of pantothenic acid production. A summary of known methods of cultivation is given in the textbook by Chmiel (Bioprozesstechnik 1. Einführung in die Bioverfahrenstechnik (Gustav Fischer Verlag, Stuttgart, 1991)) or in the text book by Storhas (Bioreaktoren und periphere Einrichtungen (Vieweg Verlag, Braunschweig/Wiesbaden, 1994)).

The culture medium to be used must comply with the requirements of the particular microorganisms in an appropriate manner. Descriptions of culture media for various microorganisms are given in the book “Manual of Methods for General Bacteriology” by the American Society for Bacteriology (Washington D.C., USA, 1981).

Sources of carbon which may be used are sugar and carbohydrates such as e.g. glucose, saccharose, lactose, fructose, maltose, molasses, starch and cellulose, oils and fats such as e.g. soy oil, sunflower oil, peanut oil and coconut fat, fatty acids such as e.g. palmitic acid, stearic acid and linoleic acid, alcohols such as e.g. glycerine and ethanol and organic acids such as e.g. acetic acid. These substances may be used individually or as a mixture.

Sources of nitrogen which may be used are organic, nitrogen-containing compounds such as peptones, yeast extract, meat extract, malt extract, corn steep liquor, soy bean flour and urea or inorganic compounds such as ammonium sulfate, ammonium chloride, ammonium phosphate, ammonium carbonate and ammonium nitrate. The sources of nitrogen may be used individually or as a mixture.

Sources of phosphorus which may be used are potassium dihydrogen phosphate or dipotassium hydrogen phosphate or the corresponding sodium-containing salts. The culture medium also has to contain salts of metals, such as e.g. magnesium sulfate or iron sulfate, which are required for growth. Finally, essential growth substances such as amino acids and vitamins may also be used in addition to the substances mentioned above. Over and above these, precursors of pantothenic acid such as aspartate, β-alanine, ketoisovalerate, ketopantoic acid or pantoic acid, and optionally their salts, may be added to the culture medium for an additional increase in pantothenic acid production. The feed materials mentioned may be added to the culture in the form of a one-off batch or may be fed in a suitable manner during cultivation.

For pH regulation of the culture, basic compounds such as sodium hydroxide, potassium hydroxide, ammonia or acid compounds such as phosphoric acid or sulfuric acid are used in an appropriate manner. To control the production of foam, antifoaming agents such as e.g. polyglycol esters of fatty acids, may be used. To maintain the stability of plasmids, suitable selective substances such as e.g. antibiotics, may be added to the medium. In order to maintain aerobic conditions, oxygen or oxygen-containing gas mixtures, such as e.g. air, are introduced into the culture. The temperature of the culture is normally 20° C. to 45° C. and is preferably 25° C. to 40° C. The culture is continued until a maximum amount of pantothenic acid has been produced. This objective is normally achieved within 10 hours to 160 hours.

The concentration of pantothenic acid can be determined using known chemical (Velisek; Chromatographic Science 60, 515-560 (1992)) or microbiological methods such as e.g. the Lactobacillus plantarum test (DIFCO MANUAL, 10^{th} Edition, p. 1100-1102; Michigan, USA).

The D-pantothenic acid can be used either in the isolated, pure form or else, together with constituents of the fermentation broth, in the solid form, in particular for animal nutrition.

If the desired concentrations of D-pantothenic acid are not achieved during fermentation, D-pantothenic acid is added in the required amount to the mixture of fermentation broth constituents and the acid.

The following microorganisms were deposited at the German Collection of Microorganisms and Cell Cultures (DSMZ, Braunschweig, Germany) in accordance with the Budapest treaty:

Escherichia coli strain DH5α/pK19mobsacBΔpck as DSM 13047

Corynebacterium glutamicum ATCC13032Δilva as DSM 12455

Corynebacterium glutamicum ATCC13022pND-D2 as DSM12438

Corynebacterium glutamicum DSM5715pEC-XT99A as DSM 12967

The present invention is explained in more detail in the following, using working examples.

For this purpose, trials were performed with the isoleucine-requiring strain ATCC13032ΔilvA. The strain ATCC13032ΔilvA has been deposited at the German Collection of Microorganisms and Cell Cultures in Braunschweig (Germany), in accordance with the Budapest treaty as DSM12455. The panD gene is described by Dusch et al. (Applied and Environmental Microbiology 65(4), 1530-1539 (1999)) and in DE 19855313.7 und has also been deposited at the German Collection of Microorganisms and Cell Cultures in Braunschweig (Germany), in accordance with the Budapest treaty in the form of the strain Corynebacterium glutamicum ATCC13032/pND-D2, as DSM12438.

### EXAMPLE 1

Isolating the pck gene

To isolate the PEP carboxykinase gene (pck) from C. glutamicum, based on cosmid pHC79 (Hohn and Collins, Gene 11 (1980) 291-298), a cosmid gene library was compiled using known methodology (Sambrook et al., Molecular Cloning, A Laboratory Handbook, 1989, Cold Spring Harbor Laboratory Press). For this purpose, chromosomal DNA was isolated from C. glutamicum ATCC13032 (Eikmanns et al., Microbiology 140 (1994) 1817-1828) and partly digested with the restriction enzyme Sau3A. After ligation of the fragments obtained into the BamHI cleavage site of the cosmid pHC79, the mixture was packaged in the protein coat of the bacteriophage lambda and the E. coli strain ED8654 (Murray et al. Molecular and General Genetics 150 (1997) 53-61) transfixed therewith. Packaging of the recombinant cosmids in the protein coat of phage lambda was performed using a method developed by Sternberg et al. (Gene 1 (1979) 255-280), the transfection of E. coli ED8654 used a method developed by Sambrook et al. (Molecular Cloning, A Laboratory Handbook, 1989, Cold Spring Harbor Laboratory Press). The corresponding cosmids were isolated from a total of 30 of the E. coli clones obtained (Sambrook et al., Molecular Cloning, A Laboratory Handbook, 1989, Cold Spring Harbor Laboratory Press) and subjected to restriction analysis with the enzyme HindIII. It was shown that 24 of the cosmids tested had inserts and that the inserts had sizes of approximately 35 kb. A total of 2200 cosmid-containing E. coli clones were combined and the cosmid DNA was prepared from this mixture, using a known process (Sambrook et al., Molecular Cloning, A Laboratory Handbook, 1989, Cold Spring Harbor Laboratory Press).

To isolate the pck gene from C. glutamicum, the cosmid gene library in PEP carboxykinase-defective E. coli mutant HG4 (Goldie and Sanwal, Journal of Bacteriology 141 (1980) 115-1121) was transformed using known processes (Sambrook et al., Molecular Cloning, A Laboratory Handbook, 1989, Cold Spring Harbor Laboratory Press). The mutant HG4, due to its lack of PEP carboxykinase, is no longer able to grow on succinate as the only source of carbon. After transformation of the cosmid gene library in this mutant a total of 1200 clones were obtained. Of these a total of two clones exhibited growth on M9 minimal medium (Sambrook et al., Molecular Cloning, A Laboratory Handbook, 1989, Cold Spring Harbor Laboratory Press) with succinate (0.4%) as the only source of carbon. After isolation of the corresponding cosmids (Sambrook et al., Molecular Cloning, A Laboratory Handbook, 1989, Cold Spring Harbor Laboratory Press) from these clones and renewed transformation in E. coli mutant HG4, the resulting clones were again able to grow on M9 medium with succinate as the only source of carbon.

In order to restrict the pck gene from C. glutamicum to a smaller fragment, the two complementary cosmids were digested with restriction enzymes XhoI, ScaI and PvuII and separated using known methods (Sambrook et al., Molecular Cloning, A Laboratory Handbook, 1989, Cold Spring Harbor Laboratory Press) in an electric field on a 0.8% agarose gel. Fragments in the size range greater than 3.0 kb were isolated from the gel by electroelution (Sambrook et al., Molecular Cloning, A Laboratory Handbook, 1989, Cold Spring Harbor Laboratory Press) and ligated into the SalI (XhoI digestion), or into the Klenow-treated EcoRI cleavage site (ScaI and PvuII digestion) of the vector pEK1 (Eikmanns et al., Gene 102 (1991) 93-98). E. coli HG4 was transformed with the ligation batches and the transformants obtained were again tested for their ability to grow on succinate as the only source of carbon. In the transformation batch with the PvuII ligation batch, seven clones appeared in which plasmids of the mutant HG4 enabled growth on succinate. The corresponding plasmids were isolated from the recombinant strains and subjected to restriction mapping. It was shown that all seven plasmids contained the same 4.3 kb PvuII insert, three in one orientation, four in the other. Depending on the orientation of the insert in the vector, the new plasmids produced were named pEK-pckA and pEK-pckB. The restriction maps of the two plasmids are given in FIGS. 1 and 2.

### EXAMPLE 2

Sequencing the pck structure gene and adjacent regions

For the sequencing procedure, the approximately 3.9 kb size EcoRI fragment from pEK-pckA (an EcoRI cleavage site arose from the vector pEK0) was isolated using known methods. The overhanging ends of the fragment were filled with Klenow polymerase to give smooth ends (Sambrook set al., Molecular Cloning, A Laboratory Handbook, 1989, Cold Spring Harbor Laboratory Press) and ligated into the EcoRV cleavage site of the vector pGEM-5Zf(+) (Promega Corporation, Madison, Wis., USA). Insertion of the plasmids produced in this way was sequenced by the chain termination sequencing method (Sanger et al., Proceedings of the National Academy of Sciences USA, 74 (1977) 5463-5467). This is given as SEQ ID No. 1. The nucleotide sequence of 3935 kb obtained was analysed using the program package HUSAR (Release 3.0) from the German Cancer Research Centre (DKFZ, Heidelberg, Germany). Sequence analysis of the fragments produced an open reading frame of 1830 kb length, which coded for a protein consisting of 610 amino acids.

### EXAMPLE 3

Preparing an integration plasmid for deletion mutagenesis of the pck gene

To inactivate the PEP carboxykinase gene, the EcoRI-SacI fragment of the pck gene was isolated from the vector pEK-pckB (FIG. 2) and inserted into the vector pGEM-7Zf (+)(Promega Corporation, Madison, Wis., USA). From the resulting plasmid, a pck-internal 1.07 kb HindII-HindIII fragment was deleted, then the pck gene with the 1.07 kb deletion was isolated as a BfrI-SacI fragment and after filling the overhanging ends ligated into the vector pK19mobsacB (Schäfer et al., Gene 145, 69-73 (1994)) which does not replicate in C. glutamicum. In the integration plasmid pK19mobsacBΔpck (FIG. 3) constructed in this way, the 5′ region of the pck gene (340 bp) is directly adjacent to the 3′ region of the pck gene (340 bp); in the genome the two regions are separated from each other by 1071 bp. Cloning was performed in E. coli DH5a as host.

### EXAMPLE 4

Deletion mutagenesis of the pck gene in the strain ATCC13032ΔilvA

To delete the pck gene, the integration plasmid pK19mobsacBΔpck was electroporated into the strain C. glutamicum ATCC13032ΔilvA. After selection on kanamycin (25 μg/ml), individual clones were obtained in which the inactivation vector was present integrated in the genome. In order to enable excision of the vector, individual colonies were incubated in 50 ml of liquid LB medium without antibiotics for 24 hours at 30° C. and 130 rpm and then painted onto saccharose-containing agar plates (LB with 15 mg/ml agar and 10% saccharose). As a result of this selection procedure, clones were obtained which had again lost the vector portion due to a second recombination event (Jäger et al. 1992, Journal of Bacteriology 174:5462-5465). In order to identify those clones in which the incomplete pck gene was now present in the genome, a polymerase chain reaction was performed. The oligonucleotides were chosen in such a way that they spanned the deletion region. The primers pck-1 with the sequence 5′-GGAACTGCTGAACTGGATCG-3′ and pck-2 with the sequence 5′-GAACTGGCTGTGAACCTCTG-3′ enhanced a 1741 bp sized fragment in the total DNA of the starting strain C. glutamicum ATCC13032ΔilvA, whereas the primers enhanced a shortened, 670 bp sized fragment in the DNA from pck deletion mutants. A deletion mutant identified in this way thus lacks the 1.07 kb size fragment of the pck gene previously deleted in vitro.

The strain C. glutamicum ATCC13032ΔilvAΔpck prepared and tested in this way was used for the further investigations.

### EXAMPLE 5

Preparing the plasmid pXT-panD

5.1 Preparing E. coli-C. glutamicum shuttle vector pEC-XT99A

The E. coli expression vector pTRC99A (Amann et al. 1988, Gene 69:301-315) was used as starting vector for constructing the E. coli-C. glutamicum shuttle expression vector pEC-XT99A. After BspHI restriction cleavage (Roche Diagnostics GmbH, Mannheim, Germany, product description BspHI, Product No. 1467123) followed by Klenow treatment (Amersham Pharmacia Biotech, Freiburg, Germany, product description Klenow fragment of DNA polymerase I, Product No. 27-0928-01; method according to Sambrook et al., 1989, Molecular Cloning: A Laboratory Manual, Cold Spring Harbor) the ampicillin resistant gene (bla) was exchanged for the tetracyclin resistant gene in C. glutamicum plasmid pAG1 (Gene Library Accession No. AF121000). For this, the resistance gene-containing region was cloned as an AluI fragment (Amersham Pharmacia Biotech, Freiburg, Germany, product description AluI, Product No. 27-0884-01) in linearised E. coli expression vector pTRC99A. Ligation was performed as described by Sambrook et al. (1989, Molecular Cloning: A Laboratory Manual, Cold Spring Harbor), wherein the DNA mixture was incubated overnight with T4 ligase (Amersham Pharmacia Biotech, Freiburg, Germany, product description T4-DNA-Ligase, Product No. 27-0870-04). This ligation mixture was then electroporated into E. coli strain DH5αmcr (Grant, 1990, Proceedings of the National Academy of Sciences U.S.A., 87:4645-4649) (Tauch et al. 1994, FEMS Microbiology Letters, 123:343-7). The E. coli expression vector produced was named pXT99A.

The plasmid pGA1 (Sonnen et al. 1991, Gene, 107:69-74) was used as the basis for cloning a minimal replicon from Corynebacterium glutamicum. By means of BalI/PstI restriction cleavage (Promega GmbH, Mannheim, Germany, product description BalI, Product No. R6691; Amersham Pharmacia Biotech, Freiburg, Germany product description PstI, Product No. 27-0976-01) of the vector pGA1, a 3484 bp size fragment can be cloned in vector pK18mob2 (Tauch et al., 1998, Archives of Microbiology 169:303-312) fragmented with SmaI and PstI (Amersham Pharmacia Biotech, Freiburg, Germany, product description SmaI, Product No. 27-0942-02, product description PstI, Product No. 27-0976-01). By using BamHI/XhoI restriction cleavage (Amersham Pharmacia Biotech, Freiburg, Germany, product description BamHI, Product No. 27-086803, product description XhoI, Product No. 27-0950-01) and subsequent Klenow treatment (Amersham Pharmacia Biotech, Freiburg, Germany, product description Klenow Fragment of DNA Polymerase I, Product No. 27-0928-01; method according to Sambrook et al., 1989, Molecular Cloning: A laboratory Manual, Cold Spring Harbor) a 839 bp size fragment was deleted. From the structure religated with T4 ligase (Amersham Pharmacia Biotech, Freiburg, Germany, product description T4-DNA-Ligase, Product No. 27-0870-04) the C. glutamicum minimal replicon could be cloned as a 2645 bp sized fragment in the E. coli expression vector pXT99A. For this, the DNA in the minimal replicon-containing structure was cleaved with the restriction enzymes KpnI (Amersham Pharmacia Biotech, Freiburg, Germany, product description KpnI, Product No. 27-0908-01) and PstI (Amersham Pharmacia Biotech, Freiburg, Germany, product description PstI, Product No. 27-0886-03) and then a 3′-5′ exonuclease treatment (Sambrook et al., 1989, Molecular Cloning: A laboratory Manual, Cold Spring Harbor) was performed using Klenow polymerase (Amersham Pharmacia Biotech, Freiburg, Germany, product description Klenow Fragment of DNA Polymerase I, Product No. 27-0928-01).

In a parallel batch, the E. coli expression vector pXT99A was cleaved with the restriction enzyme RsrII (Roche Diagnostics, Mannheim, Germany, product description RsrII, Product No. 1292587) and prepared for ligation with Klenow polymerase (Amersham Pharmacia Biotech, Freiburg, Germany, Klenow Fragment of DNA Polymerase I, Product No. 27-0928-01). Ligation of the minimal replicon with the vector structure pXT99A was performed as described by Sambrook et al. (1989, Molecular Cloning: A laboratory Manual, Cold Spring Harbor), wherein the DNA mixture was incubated overnight with T4 ligase (Amersham Pharmacia Biotech, Freiburg, Germany, product description T4-DNA-Ligase, Product No. 27-0870-04).

The E. coli-C. glutamicum shuttle expression vector pEC-XT99A produced in this way was transferred into C. glutamicum DSM5715 by means of electroporation (Liebl et al., 1989, FEMS Microbiology Letters, 53:299-303). Selection of the transformants was performed on LBHIS agar consisting of 18.5 g/l brain-heart infusion broth, 0.5 M sorbitol, 5 g/l bacto-trypton, 2.5 g/l bacto-yeast extract, 5 g/l NaCl and 18 g/l bacto-agar, which had been supplemented with 5 mg/l tetracyclin. Incubation took place for 2 days at 33° C.

Plasmid DNA was isolated from a transformant by the conventional methods (Peters-Wendisch et al., 1998, Microbiology, 144, 915-927), cleaved with the restriction endonuclease HindIII and the plasmid was then tested by agarose gel electrophoresis.

The plasmid structure obtained in this way was named pEC-XT99A and is shown in FIG. 4. The strain obtained by electroporation of the plasmid pEC-XT99A into the Corynebacterium glutamicum strain DSM5715 was named DSM5715/pEC-XT99A and was deposited as DSM12967 at the German Collection of Microorganisms and Cell Cultures (DSMZ, Braunschweig, Germany) in accordance with the Budapest treaty.

5.2 Preparing the plasmid pXT-panD

Starting from the nucleotide sequence for the pantothenate biosynthesis gene panD from C. glutamicum ATCC 13032 (Dusch et al. (Applied and Environmental Microbiology 65(4), 1530-1539 (1999)) and DE 19855313.7) PCR primers were chosen in such a way that the amplified fragment contained the gene with its native ribosome bonding site. The 405 bp size fragment amplified with the PCR primers panD-Cg1 (5′-CATCTCACGCTATGAATTCT-3′) and panD-Cg2 (5′-ACGAGGCCTGCAGCAATA-3) was then ligated, using the manufacturer's data, into the vector pCR®2.1 (Original TA Cloning Kit, Invitrogene (Leek, Netherlands), product description Original TA Cloning® Kit, Cat. no. KNM2030-01) and then transformed in the E. coli strain TOP10F′ (Catalogue “Invitrogen 2000” from the Invitrogen Co., Groningen, Netherlands). Selection of the transformants took place by incubating at 37° C. for 24 hours on LB agar plates with 100 μg/ml ampicillin and 40 μg/ml X-Gal (5-bromo-4-chloro-3-indolyl-β-D-galactoside).

DNA from the plasmid pCR-D2 built up in this way was isolated from a transformant in the conventional way, digested with the restriction endonucleases SacI and XbaI and ligated in the also cleaved vector pEC-XT99A. Since the second XbaI cleavage site, which lies within the panD coding region, is present in the methylated form in the E. coli host Top10F′, this cleavage site was not cleaved and the gene was thus cleaved out of plasmid pCR-D2 intact due to the flanking SacI and XbaI cleavage sites. After ligation, the batch was electroporated into the strain E. coli DH5αmcr. Selection took place on LB agar plates using μg/ml kanamycin. Plasmid DNA from a transformant obtained in this way was isolated, cleaved with the restriction endonucleases SacI and XbaI and the fragments were then tested by agarose gel electrophoresis. The plasmid built up in this way was named pXT-panD and is shown in FIG. 5.

### EXAMPLE 6

Preparing the pantothenic acid producers

ATCC13032ΔilvAΔpck/pXT-panD and ATCC13032ΔilvA/pXT-panD

The plasmid pXT-panD described in example 5 was electroporated into the two C. glutamicum strains ATCC13032ΔilvA and ATCC13032ΔilvAΔpck and, after selection on LB agar plates with 10 μg/ml tetracyclin, the plasmid was reisolated from each of the transformants and cleaved and tested as described in example 5.

### EXAMPLE 7

Preparing pantothenic acid

The formation of pantothenate by the C. glutamicum strains ATCC13032ΔilvA/pXT-panD and ATCC13032ΔilvAΔpck/pXT-panD was tested in medium CGXII (Keilhauer et al., 1993, Journal of Bacteriology, 175:5595-5603; table 1), which was supplemented with 10 μg/ml tetracyclin and 2 mM isoleucine.

This medium is called C. glutamicum test medium in the following. Each 50 ml of freshly made up C. glutamicum test medium was inoculated from a 16 hour old preculture of the same medium in such a way that the optical density of the culture suspension (OD₅₈₀) at the start of incubation was 0.1. The cultures were incubated at 30° C. and 130 rpm. After a 5 hour period of incubation, IPTG (isopropyl β-D-thiogalactoside) was added to give a final concentration of 1 mM. After a 48 hour period of incubation, the optical density (OD₅₈₀) of the culture was determined and then the cells were removed by centrifuging for 10 minutes at 5000 g and the supernatant liquid was filtered sterile.

TABLE 1

| | Amount | | |
|---|---|---|---|
| Substance | per liter | | Comment |
| (NH₄)₂ SO₂ | 20 | g | |
| Urea | 5 | g | |
| KH₂PO₄ | 1 | g | |
| K₂HPO₄ | 1 | g | |
| MgSO₄ * 7 H₂O | 0.25 | g | |
| MOPS | 42 | g | |
| CaCl₂ | 10 | mg | |
| FeSO₄ * 7 H₂O | 10 | mg | |
| MnSO₄ * H₂O | 10 | mg | |
| ZnSO₄ * 7 H₂O | 1 | mg | |
| CuSO₄ | 0.2 | mg | |
| NiCl₂ * 6 H₂O | 0.02 | mg | |
| Biotin | 0.5 | mg | |
| Glucose | 40 | g | autoclave |
| | | | separately |
| Protocatechuic | 0.03 | mg | filter |
| acid | | | sterile |

To determine the optical density, a Novaspec II Photometer from the Pharmacia Co. (Freiburg, Germany) was set to a measurement wavelength of 580 nm.

Quantification of the D-panthothenate in the culture supernatant liquid was performed using Lactobacillus plantarum ATCC 8014 according to data in the manual from the DIFCO Co. (DIFCO MANUAL, 10^{th} Edition, p. 1100-1102; Michigan, USA). For calibration purposes, the hemicalcium salt of pantothenate from Sigma Co. (Deisenhofen, Germany) was used.

The results of pantothenate production by the strains ATCC13032ΔilvA/pXT-panD and ATCC13032ΔilvAΔpck/pXT-panD are given in Table 2.

TABLE 2

| | Cell | |
|---|---|---|
| | density | Concentration |
| Strain | OD₅₈₀ | (ng/ml) |
| ATCC13032ΔilvA/pXT-panD | 16.0 | 10.4 |
| ATCC13032ΔilvAΔpck/pXT-panD | 17.0 | 34.9 |

### BRIEF DESCRIPTION OF THE FIGURES

FIG. 1: Restriction map of the plasmid pEK-pckA

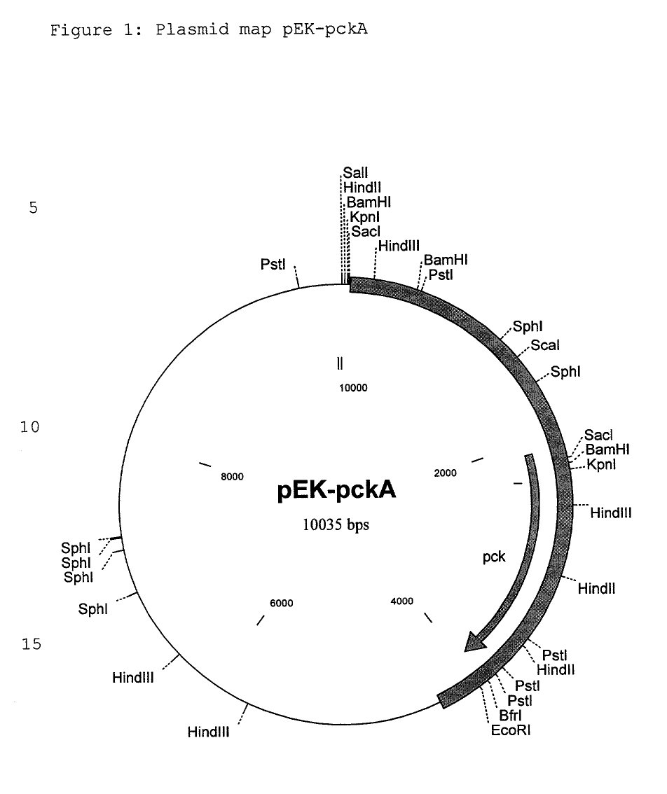

FIG. 2: Restriction map of the plasmid pEK-pckB

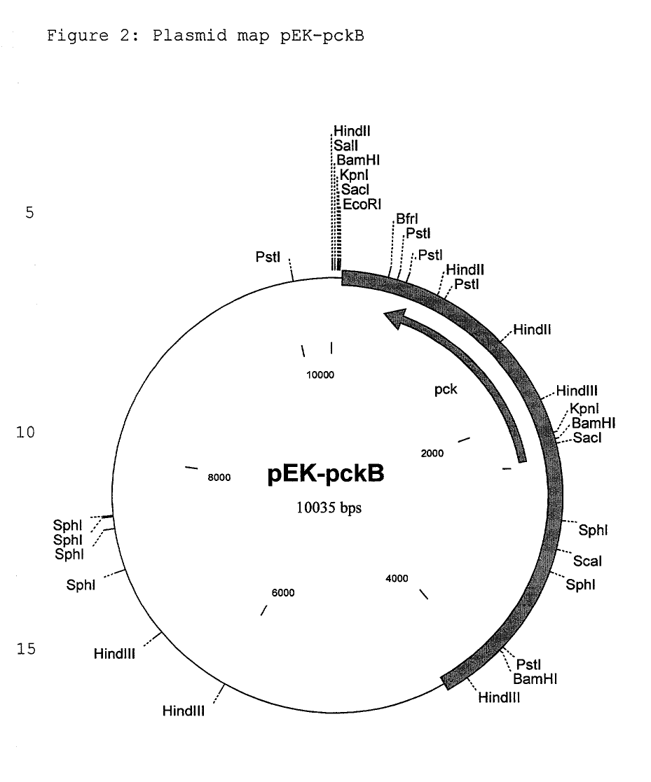

FIG. 3: Restriction map of the plasmid pK19mobsacBΔpck

FIG. 4: Restriction map of the plasmid pEC-XT99A

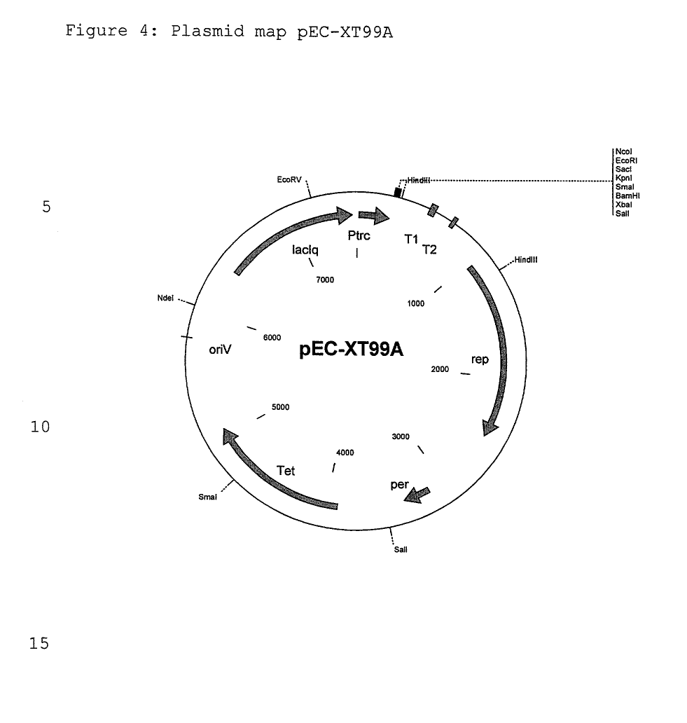

FIG. 5: Restriction map of the plasmid pXT-panD.

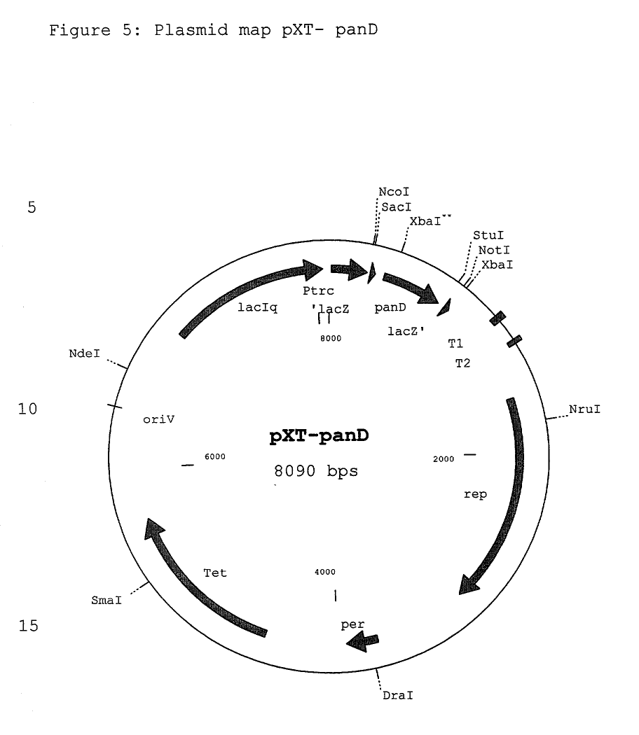

The data given for numbers of base pairs are approximate values which are obtained in the context of reproducibility.

A key to the abbreviations and names used is given below:

| | |
|---|---|
| ′lacZ: | 3′-terminus of the lacZα gene fragment |
| Km-r: | Kanamycin resistance gene |
| lacIq: | LacIq allele of the lac repressor gene |
| lacZ′: | 5′-terminus of the lacZα gene fragment |
| oriT: | Replication origin for transfer |
| oriV: | Replication origin V |
| panD: | Aspartate decarboxylase gene |
| pck: | pck gene |
| pck′: | 3′-terminales Fragment des pck gene |
| pck″: | 5′-terminal fragment of the pck gene |
| per: | Gene to regulate the copy number |
| Ptrc: | trc promotor |
| rep: | Replication region for C. glutamicum |
| sacB: | sacB gene |
| T1: | Transcription terminator T1 |
| T2: | Transcription terminator T2 |
| Tet: | Tetracyclin resistance gene |
| BamHI: | Cleavage site for restriction enzyme BamHI |
| BfrI: | Cleavage site for restriction enzyme BfrI |
| EcoRI: | Cleavage site for restriction enzyme EcoRI |
| HindII: | Cleavage site for restriction enzyme HindII |
| HindIII: | Cleavage site for restriction enzyme HindIII |
| KpnI: | Cleavage site for restriction enzyme KpnI |
| NcoI: | Cleavage site for restriction enzyme NcoI |
| NotI: | Cleavage site for restriction enzyme NotI |
| PstI: | Cleavage site for restriction enzyme PstI |
| SacI: | Cleavage site for restriction enzyme SacI |
| SalI: | Cleavage site for restriction enzyme SalI |
| ScaI: | Cleavage site for restriction enzyme ScaI |
| SmaI: | Cleavage site for restriction enzyme SmaI |
| SphI: | Cleavage site for restriction enzyme SphI |
| XbaI**: | Methylated cleavage site XbaI |
| XbaI: | Cleavage site for restriction enzyme XbaI |

What is claimed is:

1. A process for preparing D-pantothenic acid by the fermentation of coryneform bacteria, wherein bacteria are used in which the nucleotide sequence (pck) coding for phosphoenolpyruvate carboxykinase (PEP carboxykinase) (EC 4.1.1.49) is attenuated, in particular switched off.

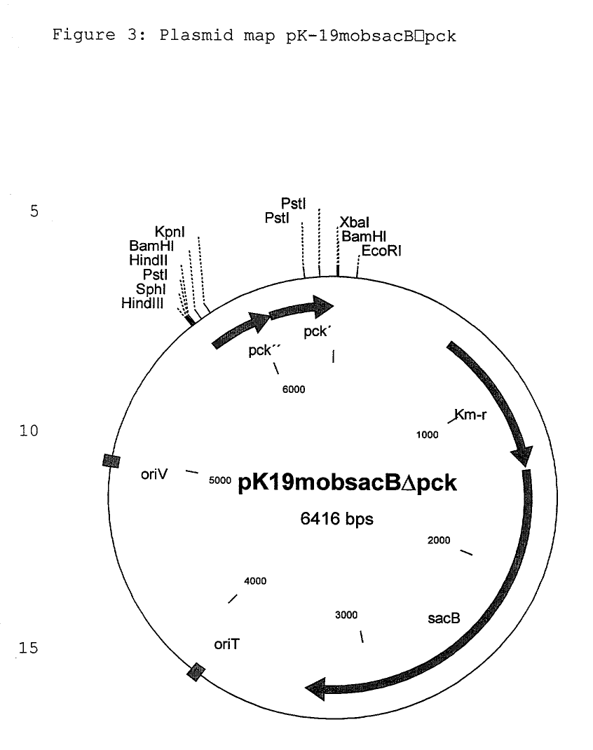

2. A process according to claim 1, wherein, to produce attenuation, the process of deletion of the pck gene is used, in particular using the vector pk19mobsacBΔpck, shown in FIG. 3 and deposited in E. coli as DSM 13047.

3. A process according to claim 1, wherein bacteria are used in which in addition genes in the biosynthetic pathway for D-pantothenic acid are enhanced.

4. A process according to claim 1, wherein bacteria are used in the which the metabolic pathways which reduce the formation of D-pantothenic acid are at least partly switched off.

5. A process according to claim 3, wherein the panB gene coding for ketopantoate hydroxymethyltransferase is simultaneously enhanced.

6. A process according to claim 3, wherein the panC gene coding for pantothenate synthetase is simultaneously enhanced.

7. A process according to claim 3, wherein the ilvD gene coding for dihydroxy acid dehydratase is simultaneously enhanced.

8. A process according to claims 5 to 7, wherein the genes mentioned are enhanced in coryneform bacteria which already produce pantothenic acid.

9. A process for the fermentative preparation of D-pantothenic acid in accordance with one or more of the preceding Claims, wherein the following steps are performed:
a) fermentation of D-pantothenic acid producing bacteria in which at least the gene coding for phosphoenolpyruvate carboxykinase is attenuated,
b) enrichment of D-pantothenic acid in the medium or in the cells of the bacteria, and
c) isolation of the D-pantothenic acid produced.

10. Coryneform bacteria in which the nucleotide sequences (pck gene) coding for phosphoenolpyruvate carboxykinase (PEP carboxykinase) are attenuated.

11. Escherichia coli strain DH5α/pK19mobsacBΔpck, deposited under the number DSM 13047 at DSMZ, Braunschweig.
